(19) United States
(12) Patent Application Publication
(10) Pub. No.: US 2006/0106226 A1
(43) Pub. Date: May 18, 2006

(54) Isolation of amino acids and related isolates

(75) Inventors: Robert L. Pollack, Philadelphia, PA (US); John C. Godfrey, Huntingdon Valley, PA (US); Sharon Gaglia, Veneta, OR (US)

Correspondence Address: SCHWABE, WILLIAMSON & WYATT, P.C.;PACWEST CENTER, SUITE 1900, 1211 SW FIFTH AVENUE, PORTLAND, OR, 97204, US

(73) Assignee: AMINOPATH LABS, LLC and a Patent License Agreement

(21) Appl. No.: 11/330,572

(22) Filed: Jan. 11, 2006

Related U.S. Application Data
(63) Continuation-in-part of application No. 10/367,667, filed on Feb. 14, 2003.
Application No. 10/367,667 is a continuation-in-part of application No. 09/924,387, filed on Aug. 7, 2001, now Pat. No. 6,541,644.
Application No. 09/924,387 is a continuation of application No. 09/361,489, filed on Jul. 26, 1999, now abandoned.
Application No. 09/361,489 is a continuation of application No. 09/030,952, filed on Feb. 26, 1998, now Pat. No. 5,945,542.
(60) Provisional application No. 60/362,933, filed on Mar. 7, 2002.

(51) Int. Cl. C07C 227/40; C07D 233/61; C07D 209/20

(52) U.S. Cl. 548/498; 562/443; 562/444; 548/335.5

(57) ABSTRACT

Embodiments of the present invention provide processes for extracting amino acids from mixtures of amino acids, and compositions and mixtures formed therefrom. Applications include separating and/or isolating non-aromatic amino acids, and/or separating aromatic amino acids into at least one fraction containing phenylalanine and tyrosine and a fraction containing L-β-3-indolylalanine (L-β-3) and providing natural or other amino acid mixtures thereof. The source of amino acids may include a natural source, such as an enzymaticaly hydrolyzed protein or other natural protein hydrolysates containing mixtures of free amino acids. The processes of embodiments of the present invention include contacting the mixture of amino acids with a resin or hydrophobic substance that is attractive to aromatic amino acids but not attractive to aliphatic amino acids to separate the aromatic amino acids from the rest of the mixture.

### CROSS REFERENCE TO RELATED APPLICATIONS

The present application is a continuation-in-part of and claims priority to U.S. patent application Ser. No. 10/367,667, filed on Feb. 14, 2003, entitled, “Isolation of Natural L-β-3-Indolylalanine and Enrichment of Natural Aliphatic Amino Acid Mixtures with Natural L-β-3-lndolylalanine,” which is a continuation-in-part of similarly entitled U.S. patent application Ser. No. 09/924,387, filed on Aug. 7, 2001, and issued on Apr. 1, 2003, as U.S. Pat. No. 6,541,644, which is a continuation of U.S. patent application Ser. No. 09/361,489, filed on Jul. 26, 1999, now abandoned, which is a continuation of U.S. patent application Ser. No. 09/030,952, filed on Feb. 26, 1998, and issued on Aug. 31, 1999, as U.S. Pat. No. 5,945,542. U.S. patent application Ser. No. 10/367,667 also claims priority to U.S. Provisional Patent Application No. 60/362,933, filed on Mar. 7, 2002. The entire contents and disclosures of the above-identified patent applications are hereby incorporated by reference in their entirety.

### TECHNICAL FIELD

Embodiments of the present invention relate to the field of biochemical processing, more specifically, to the extraction of amino acids and related isolates from sources or mixtures of amino acids.

### BACKGROUND

Extracting beneficial elements, such as amino acids, from natural sources is an important mechanism, for example, to provide for natural dietary supplements or components thereof. Attempts made to isolate various amino acids have been mostly unsuccessful. Prior to 1989, for example, L-β-3 indolylalanine, hereafter L-β-3, was available to consumers as a dietary supplement and could be purchased freely. Studies on the oral administration of L-β-3 under proper dietary conditions that provided a supplementary intake of this particular amino acid showed that supplemental L-β-3 helped to correct an improper L-β-3/LNM ratio in the brain.

In the late 1980's, all of the L-β-3 used in the United States was imported from Japan. In 1989, the U.S. Food and Drug Administration (FDA) halted the importation and sale of L-β-3 in the U.S. as a result of a highly toxic contaminant that was found in batches of L-β-3 made by a bacterial fermentation process used to produce L-β-3. To date, the importation of L-β-3 into the U.S. for human consumption and the sale of such imported L-β-3-containing products has not resumed.

Thus, safe mechanisms for obtaining such amino acids, and related isolates, are still largely unavailable.

### BRIEF DESCRIPTION OF THE DRAWINGS

Embodiments of the present invention will be readily understood by the following detailed description in conjunction with the accompanying drawings. To facilitate this description, like reference numerals designate like structural elements. Embodiments of the invention are illustrated by way of example and not by way of limitation in the figures of the accompanying drawings.

FIG. 1 illustrates a process flow diagram in accordance with various embodiments of the present invention; and

FIG. 2 shows exemplary process conditions for an embodiment of the present invention.

### DETAILED DESCRIPTION OF EMBODIMENTS OF THE INVENTION

In the following detailed description, reference is made to the accompanying drawings which form a part hereof wherein like numerals designate like parts throughout, and in which is shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete operations in turn, in a manner that may be helpful in understanding embodiments of the present invention; however, the order of description should not be construed to imply that these operations are order dependent.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

Embodiments of the present invention are directed to processes, and compositions based on compounds obtained by such processes, for the extraction of amino acids and related isolates from sources of or mixtures of amino acids, including mixtures of naturally occurring amino acids.

Embodiments of the present invention are directed generally to the extraction of amino acids from sources of, or mixtures of, amino acids, such as naturally occurring mixtures of amino acids.

An embodiment of the present invention relates to the extraction, isolation or separation of specific amino acids, or multiple amino acids, based on properties of the amino acids.

An embodiment of the present invention relates to the selective isolation of non-aromatic amino acids, phenylalanine, tyrosine, and/or L-β-3. For the purposes of the present invention, the term “selective isolation” refers to the separation of an amino acid source such that a fraction of an amino acid source targeted for isolation of one or more amino acids will have a greater amount by weight of the one or more targeted amino acids than is present in the other fractions.

An embodiment of the present invention includes separation of the amino acid L-β-3 from a natural source of a mixture of amino acids, for example an enzymaticaly hydrolyzed protein or other natural protein hydrolysates containing mixtures of free amino acids; preparation of an amino acid fraction from the aforementioned L-β-3 and an amino acid mixture (obtained during the aforementioned separation) that is substantially or completely free of aromatic amino acids, particularly phenylalanine; and preparation of highly enriched mixtures of L-β-3 and one or more non-aromatic amino acids, i.e., mixtures having a concentration of L-β-3 in an amount greater than that which occurs naturally. The L-β-3 and amino acid mixtures containing L-β-3 may be used to provide dietary therapeutic supplements for increasing the production of serotonin within the brain, thereby decreasing or eliminating undesirable physiological conditions brought about by a decreased brain serotonin level.

In an embodiment of the present invention, a fraction may have less than approximately 0.05%, for example less than 0.02% or 0.03%, by weight phenylalanine on a dry weight basis and be considered substantially free of phenylalanine.

An embodiment of the present invention includes (1) the separation, as a group, of aromatic amino acids, including L-β-3, from an amino acid mixture, the mixture being obtained, for example, by the enzymatic hydrolysis of common proteins; (2) the removal of L-β-3 from the mixture of amino acids obtained in (1); and (3) producing mixtures of one or more non-aromatic amino acids with the recovered L-β-3 in various proportions. Components isolated or recovered in the processes according to embodiments of the present invention may be provided in a form suitable for further use.

Embodiments of the present invention include a process whereby a mixture of natural amino acids containing all or substantially all of the natural amino acids (i.e., glycine, the L-forms of alanine, arginine, asparagine, aspartic acid, cysteine, glutamic acid, glutamine, histidine, hydroxyproline, L-β-3, isoleucine, leucine, lysine, methionine, phenylalanine, proline, serine, threonine, tyrosine, and valine) in the form of free amino acids is separated into at least three fractions containing, respectively, (a) substantially all of the non-aromatic amino acids originally present, i.e., alanine, arginine, asparagine, aspartic acid, cysteine (in equilibrium with the dimeric cystine), glutamic acid, glutamine, glycine, histidine, hydroxyproline, isoleucine, leucine, lysine, methionine, proline, serine, threonine, and valine; (b) the monocyclic aromatic amino acids phenylalanine and tyrosine; and (c) L-β-3, the only amino acid of the group that possesses as a part of its structure two fused aromatic rings. In an embodiment of the present invention, the process may further provide a range of mixtures of fraction (a) with fraction (c) while specifically eliminating fraction (b), which may be deleterious to the desirable physiological actions of L-β-3. These mixtures including one or more aliphatic amino acids and L-β-3 are possessed of highly desirable physiological properties useful in the therapeutic relief of human suffering related to and caused, at least in part, by a relative deficiency of L-β-3. Other mixtures, with or without L-β-3, may be provided as desired in accordance with embodiments of the present invention.

In embodiments of the presentation, the separation processes do not result in perfectly separated fractions, but rather a substantial portion of a targeted amino acid is directed into the desired fraction as a result of a process in accordance with an embodiment of the present invention. For example, according to an embodiment of the present invention, an amino acid, such as isoleucine, may be present in fraction 1 and fraction 2, but comparatively, the amount of isoleucine in fraction 1, as measured by percent by weight of the fraction, is higher than in fraction 2.

In embodiments of the present invention, the source mixture of amino acids may contain fewer than all of the natural amino acids.

In one embodiment, the present invention involves applying or exposing by contact a solution of phenylalanine, tyrosine, and L-β-3 in the presence of mixed aliphatic amino acids to a hydrophobic substance such that the mixed aromatic amino acids adsorb selectively to the hydrophobic substance and may thereafter themselves be selectively and sequentially desorbed. As an illustrative example, a natural amino acid-containing mixture, or source, preferably an enzymaticaly treated or other natural protein hydrolysate, is dissolved, and contacted with a hydrophobic substance in order that aromatic amino acids may be selectively attracted to the hydrophobic substance and aliphatic amino acids may be carried away in the fluid carrier. The hydrophobic substance may thereafter be washed to remove residual non-aromatic or aliphatic amino acids, which, while having essentially no affinity for the hydrophobic substance, may be physically associated with, but not bound by attractive forces to, the hydrophobic substance.

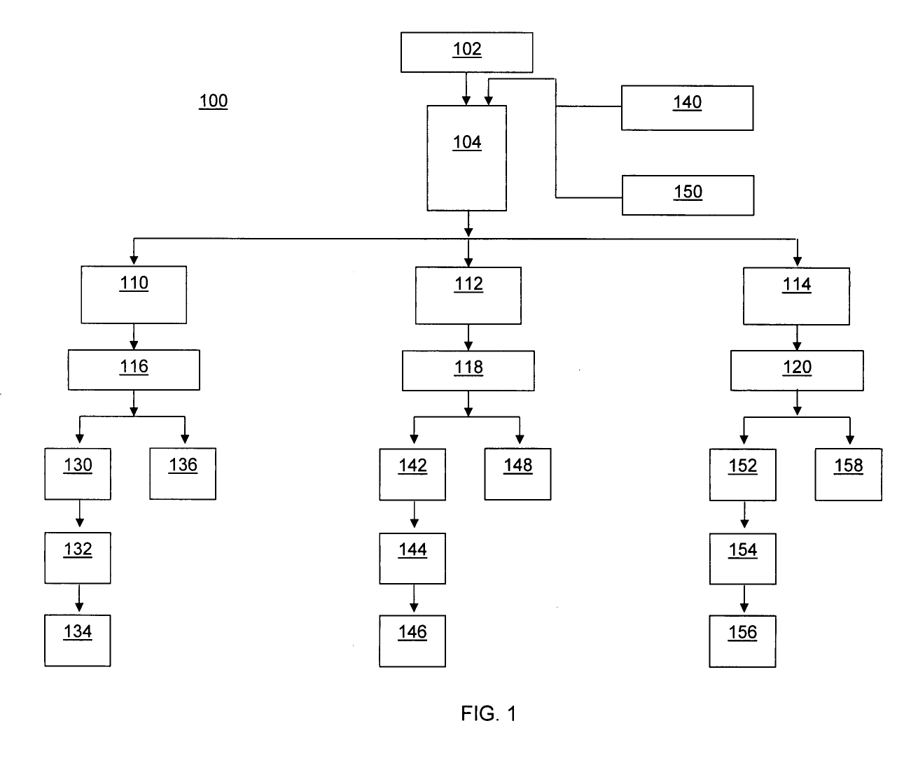

FIG. 1 illustrates a process flow diagram 100 in accordance with various embodiments of the present invention. Process flow 100 provides an exemplary process in which an initial source 102, such as an amino acid source, mixture of amino acids, hydrolysate, such as a protein hydrolysate, for example of casein, whey, or soy protein, etc., whether enzymaticaly hydrolyzed, acid hydrolyzed, etc., is introduced into a column 104 for separation into various fractions 110, 112, 114. Other natural protein hydrolysates may also be used without departing from the scope of embodiments of the present invention.

Hydrolysate may be initially made, for example, by enzymaticaly hydrolyzing a natural protein to break the protein into smaller peptides and amino acids. In an embodiment of the present invention, a solution may be made from a hydrolysate. For example, a solution may be prepared from dried hydrolysate to make a percent solids solution which may then be used as the source for a process in accordance with an embodiment of the present invention.

Column 104 may be a single column, or may be multiple columns coupled in series, such as designed for column chromatography. In embodiments of the present invention, column 104 may be loaded with a resin, such as a hydrophobic resin. A hydrophobic resin may be beneficial in that it is selectively attractive to aromatic amino acids, in comparison to non-aromatic amino acids. In embodiments, a resin may be polymeric and/or non-ionic.

When a hydrophobic resin is contacted under certain conditions with a protein source containing both aromatic and non-aromatic (aliphatic) amino acids, the aromatic amino acids will preferentially adsorb to the resin. Thus, a fraction 110 may result containing non-aromatic amino acids. Fraction 110 may be beneficial in that it may contain alanine, arginine, asparagine, aspartic acid, cysteine (in equilibrium with the dimeric cystine), glutamic acid, glutamine, glycine, histidine, hydroxyproline, isoleucine, leucine, lysine, methionine, proline, serine, threonine, and/or valine, depending on the contents of the original source. Furthermore, fraction 110 may be beneficial in that it is free of or substantially free of L-phenylalanine, which may have toxic or harmful effects especially for individuals with phenylketonuria.

In an embodiment of the present invention, an aqueous amino acid source may be passed over a resin at a particular flow rate, such as 2-5 gallons/minute, for example 3-3.5 gallons/minute. In an embodiment of the present invention, a wash agent 140, such as water or deionized water or an acid, may be used to further move the source through the column, and/or may be applied to the resin to assist in removing any residual non-aromatic amino acids. In an embodiment of the present invention, a wash agent may be provided at a similar flow rate to the flow rate at which the amino acid source was introduced to the column, or at an alternative flow rate.

After it is collected, fraction 110 may be concentrated (116), for example, by membrane filtration, reverse osmosis, nanofiltration or any other suitable mechanism for concentrating an aqueous fraction, to create a concentrate 130 and a filtrate 136. Concentrate 130 may be further processed (132), such as by drying, for example by spray drying, to create a final processed or dried fraction 134, such as in the form of a flowable powder.

In an embodiment of the present invention, after a hydrophobic resin has been contacted with a source of amino acids, aromatic amino acids may remain adsorbed to the resin. In an embodiment of the present invention, a wash agent 140 may be passed over a resin at a particular flow rate, such as 8-11 gallons/minute, for example 9.6-10.0 gallons/minute, to remove L-phenylalanine and L-tyrosine, if present in the source, to create fraction 112. In accordance with an embodiment of the present invention, a resin may be washed (once or serially) to selectively desorb phenylalanine and tyrosine from the hydrophobic substance while leaving L-β-3 adsorbed to the hydrophobic substance.

In an embodiment of the present invention, a wash agent 140 may be an acid, for example, a dilute solution of an acid, to remove L-phenylalanine and L-tyrosine, if present in the source, to create fraction 112. In an embodiment of the present invention, the hydrophobic substance may be washed with an acid capable of selectively desorbing phenylalanine and tyrosine from the hydrophobic substance while leaving L-β-3 adsorbed to the hydrophobic substance.

Fraction 112 may be concentrated (118), for example, by membrane filtration, reverse osmosis, nanofiltration or other suitable mechanism for concentrating an aqueous fraction, to create a concentrate 142 and a filtrate 148. Concentrate 142 may be further processed (144), such as by drying, for example by spray drying, to create a final processed or dried fraction 146, such as in the form of a flowable powder.

Thereafter, the hydrophobic substance may be washed (once or serially) with a suitable release agent 150 to displace L-β-3 from the hydrophobic substance. Suitable release agents for use in embodiments of the present invention may dissociate L-β-3 from the hydrophobic substance. In an embodiment of the present invention, a release agent 150 may be passed over a resin at a particular flow rate, such as 8-11 gallons/minute, for example 9.6-10.0 gallons/minute, to remove L-β-3, if present in the source, to create fraction 114.

Fraction 114 may be concentrated (120), for example, by membrane filtration, reverse osmosis, nanofiltration or other suitable mechanism for concentrating an aqueous fraction, to create a concentrate 152 and a filtrate 158. Concentrate 152 may be further processed (154), such as by drying, for example by spray drying, to create a final processed or dried fraction 156, such as in the form of a flowable powder.

In embodiments of the present invention, a suitable release agent may be isopropyl alcohol, such as a 10% isopropyl alcohol solution.

In an embodiment of the present invention, the release agent may be a base, such as a dilute base, to displace the L-β-3 from the hydrophobic substance.

In an embodiment of the present invention, after a release agent is applied, a further wash operation may be performed, such as by flushing the resin with water, for example, deionized water. In embodiments of the present invention, such a wash operation may be performed at a particular flow rate, such as 8-11 gallons/minute, for example 9.6-10.0 gallons/minute.

In embodiments of the present invention, the flow rates may be utilized to specifically remove the targeted amino acids. For example, adding a release agent at a flow rate of less than 1 gallon per minute may be insufficient in a 25 ft³ container. However, adding a release agent at a flow rate of 9 gallons per minute in a 25 ft³ container may be suitable to remove L-β-3 as discussed above.

In embodiments of the present invention, flow rates may be controlled, in accordance with the teachings herein, to provide the desired separation and operating parameters.

In embodiments of the present invention, exemplary flow rates, such as 8-11 gallons per minute for addition of a release agent, may be matched to or adjusted for the column(s) or other container(s). Flow rates may thus be selected to perform the desired separation as discussed herein, and, in embodiments, may be adjusted to maintain a suitable contact or retention time in the column(s)/container(s). Embodiments of the present invention thus work with various sizes and/or configurations of columns in accordance with the teachings herein. In an embodiment of the present invention, a flow rate may be defined per unit volume of the column(s) or container(s) and further may be volume normalized. For the purposes of the present invention, the term “volume normalized” when used with respect to a flow rate means that the flow rate is selected based on at least the volume of the cylinder.

In an embodiment of the present invention, concentrate 152 may be processed by crystallization. For example, glacial acetic acid, or other suitable solution, may be added to concentrate 152 to create a solution, such as a 25% solution of acetic acid. The solution may then be cooled to decrease solubility of L-β-3. The liquid may, in embodiments, be seeded with crystals to promote crystallization. The crystals may be further purified, in embodiments, by re-dissolving the crystals and contacting with activated carbon, and then re-crystallized. In an embodiment of the present invention, the crystals may then be dried, such as by using an air dryer or freeze dryer.

The protein hydrolysate used according to embodiments of the present invention may be “concentrated” in the sense that a higher amount of protein hydrolysate may be present in the amount of water when put in solution. The protein hydrolysate may be present, for example, in an amount of approximately 1-30% by weight of the aqueous solution containing the protein hydrolysate. A preferred range for embodiments of the present invention may be approximately 5 to 16% by weight. Most food-acceptable protein sources contain L-β-3 at about 0.5 to 1.5% by weight of the contained protein. As applied to extracting L-β-3, processes described herein may serve to concentrate the L-β-3 from sources to a range of about 10% to about 75%.

According to embodiments of the present invention, the hydrophobic substance may be, for example, a resin, or a reverse phase silica gel. In embodiments of the present invention, the substance may have an attraction for aromatic rings of amino acids but little or no attraction to aliphatic amino acids at the natural pH of the solution. The attraction to the aromatic rings of amino acids is believed to be based on the polymeric resin having attractive van der Waals interaction due to the pi-electrons of the polymer with the pi-electrons of the aromatic rings of the amino acids. The hydrophobic substance may be a porous, wettable polymeric resin, such as a non-ionic cross-linked polystyrene. A preferred polymeric resin suitable for use in the present invention is a non-ionic cross-linked polystyrene such as AMBERLITE® XAD-4 resin sold by Rohm & Haas Company. Other polymeric resins also sold by Rohm & Haas which are suitable for use include, but are not limited to, the following: AMBERLITE® XAD-16, AMBERLITE® XAD 1180, AMBERLITE® XAD-2000, AMBERLITE® XAD-2010, DIAION™ HP20, DIAION™ HP20SS, SEPABEADS™ SP20MS, AMBERCHROM® CG-71, AMBERCHROM® CG-161, AMBERCHROM® CG-300, AMBERCHROM® CG-1000, AMBERSORB® 563, AMBERSORB® 575, AMBERSORB® 348F, and AMBERSORB® 572. Other similar polymeric resins may be used as well. The hydrophobic substance may be present in any suitable form. In embodiments of the present invention, a particulate form, including the form of porous beads, for example, may be effective.

In accordance with an embodiment of the present invention, a hydrophobic substance may be contained within a porous carrier or container, which may then be exposed to an amino acid solution. A porous container may take any suitable form to contain a hydrophobic substance and to permit amino acids to permeate or otherwise flow into the container and interact with the hydrophobic substance. An example of a suitable container may be, for example, a porous container through which the mixture of amino acids may flow, yet within which the resin or other hydrophobic substance may be retained. An example of such a container may be a mesh bag, such as a fine mesh nylon bag, although other containers may be used without departing from the scope of the present invention.

Containers or columns in accordance with embodiments of the present invention may be, for example, stainless steel, and may be fitted with suitable screens, end flanges, fittings, valves, such as a flow control valve, pumps, such as a variable speed pump, flow meters, etc. to allow for operation of methods in accordance with embodiments of the present invention. In an embodiment of the present invention, a pressure regulator may be provided on the outflow of a column to provide back pressure to the liquid, for example, under a pressure of approximately 20 PSIG (pounds per square inch gauge). Back pressure helps prevent gas bubble formation in the column. In an embodiment of the present invention, a variable speed positive displacement pump may be provided on the column inlet to control the input flow rates.

Suitable acids for use in embodiments of the present invention include, but are not limited to, acetic acid, formic acid, propionic acid, butyric or isobutyric acid, and other weak acids. Suitable acids include short chain aliphatic acids having molecular weights no greater than 88.10 daltons and a Ka in the range of 1.77×10⁻⁴ and 1.34×10⁻⁵ (pKₐ in the range of 3.75 and 4.87) at 25° C. In embodiments, the acid or mixture of acids may be applied in dilute form. A preferred acid from the standpoint of function and economy is dilute acetic acid. The hydrophobic substance may then, in embodiments, be washed with water or another suitable rinsing agent to remove residual acid.

Suitable bases for use in embodiments of the present invention include, but are not limited to, ammonia (in the form of ammonium hydroxide), methylamine, dimethylamine, trimethylamine, ethylamine, diethylamine, triethylamine, and other weak bases. Suitable bases include ammonia and short chain aliphatic primary, secondary or tertiary amines having molecular weights no greater than 101.19 daltons and a Kb in the range of 1.26×10⁻³ and 1.8×10⁻⁵ (pK_{b} in the range of 2.90 and 4.74) at 25° C. The base or mixtures of bases may be applied, for example, in dilute form. According to an embodiment of the present invention, a preferred base from the standpoint of function and economy may be dilute ammonium hydroxide. Some bases may leave a salt or other residue in the final product that may be removed later, for example, in an additional rinse step. The hydrophobic substance may, in embodiments, be subjected to additional wash steps, such as with a 50-50 or other mixture of ethanol and water, to ensure full removal of L-β-3 absorbed to the substance.

In embodiments of the present invention, the recovered amino acids, such as L-β-3, may be purified from impurities including isopropyl alcohol, water, residual acid, base, or salts by any suitable purification step. For example, when an acid or base forms weak salts with amino acids, as is the case with both acetic acid and ammonium hydroxide, the salts may be volatile and readily decompose upon gentle heating. Thus, in such a case, because amino acids do not decompose upon gentle heating, the solutions may readily be heated, such as under vacuum, to remove water and either an acid (from the phenylalanine plus tyrosine fraction) or a base (from the L-β-3 fraction) leaving behind the free amino acids in substantially pure form. This evaporative concentration, when performed on an L-β-3-containing solution, provides a dry, non-hygroscopic powder while removing excess base.

In embodiments of the present invention, the L-β-3 may also be recovered by crystallization, filtration, and/or centrifugation.

In embodiments of the present invention, it may be advantageous to remove phenylalanine from L-β-3 since phenylalanine is strongly competitive with L-β-3 in the key systems which transport L-β-3 to the brain. Thus, when delivered, for example, in a dietary supplement, isolated L-β-3 may be provided in elevated levels to provide a competitive transport advantage with respect to other amino acids, such as phenylalanine.

The contacting and elutions described in embodiments of the present invention may be enabled through any suitable batch or flow-through mechanism. For example, the resin or other hydrophobic substance may be packed in a column, or a series of columns, or retained in a reservoir through which the liquids may be passed. A reservoir may retain a hydrophobic substance in a packed configuration or in a loose configuration in which the substance may move freely within the reservoir. As another example, a porous carrier containing the hydrophobic substance may be placed inside a reservoir containing the amino acid solution. In an embodiment of the present invention, the reservoir may then be agitated, by shaking, stirring, swirling, rotating or other mechanisms, to allow for rapid interaction between the hydrophobic substance and the contents of the reservoir. The reservoir may be emptied and serially refilled with the various other solutions, i.e., rinsing solutions, wash agents, release agents, acidic solutions, basic solutions, etc., with the carrier remaining inside the reservoir. Alternatively, a series of reservoirs may be used and the carrier may be transported from one reservoir to another.

Embodiments of the present invention may be further understood and described by the following examples that serve to illustrate, but not limit, embodiments of the present invention.

### EXAMPLE 1

### Fractionation of a Digest of Casein which Contains L-β-3

This example describes a general procedure for the preparation of fractions containing (a) substantially all of the non-aromatic amino acids originally present in a source containing a mixture of natural amino acids, i.e., alanine, arginine, asparagine, aspartic acid, cysteine (in equilibrium with the dimeric cystine), glutamic acid, glutamine, glycine, histidine, hydroxyproline, isoleucine, leucine, lysine, methionine, proline, serine, threonine, and valine; (b) the monocyclic amino acids phenylalanine and tyrosine; and (c) L-β-3.

A glass column, having the dimensions of 1 cm (inner diameter) by 30 cm and containing approximately 24 grams of AMBERLITE® XAD-4 (a non-ionic cross-linked polystyrene adsorbent in the form of white insoluble beads having an average diameter of 0.30 to 0.45 mm), was prepared according to the resin manufacturer's (Rohm and Haas Company) general directions for carrying out the adsorptive separation of aromatic from non-aromatic organic compounds. Briefly, the column was loosely wet-packed by pouring a suspension of the resin beads in water into the column. The column was then arranged to backwash (bottom to top) with water at a rate sufficient to expand the apparent column volume by 50%. Backwash with water was continued for ten minutes. The flow was then stopped and the resin beads were allowed to settle under the influence of gravity only, in order to achieve uniform packing of the column. After passing approximately 25 ml of water in the downward direction, down-wash was continued with 108 ml of 91% (weight/volume, aqueous) isopropyl alcohol in 37.5 minutes. The alcohol wash was followed by a wash with 432 ml of water during 1.25 hours, at an approximate flow rate of 9.6 ml/min. The resin column thus prepared was ready for the adsorptive separation of the mixed, free amino acids.

The starting material for the adsorptive separation of the mixed free amino acids had the following characteristics: a refined, enzymatic hydrolysate of casein as a dry powder containing 80% free amino acids, the remainder being almost entirely residual peptides. In terms of amino acid composition, the hydrolysate contained 819.5 mg/gm (milligrams/gram) of non-aromatic amino acids, 40.9 mg/gm of L-phenylalanine plus L-tyrosine, and 5.7 mg/gm of L-β-3.

A clear solution of the above starting material was prepared from 7.800 gm of the hydrolysate in 78 ml of water at room temperature. This solution was applied to the resin column in five portions over 20 minutes at a flow rate of 4.0 ml/min. Elution with plain water was continued until 341 ml of eluate was collected in 3 yellow fractions of 104 ml, 124 ml, and 113 ml. Evaporation to dryness of these first 3 fractions yielded, respectively, 6.578 gm, 1.016 gm, and 0.107 gm. Thin layer chromatography (TLC) demonstrated that these 3 fractions contained only the non-aromatic amino acids, i.e., they contained no L-phenylalanine, L-tyrosine, or L-β-3. Elution was then continued with 150 ml of 2% acetic acid in water, followed by 150 ml of water, and four fractions totaling 287 ml were collected. On evaporation to dryness, the fractions contained, respectively, 70.1 mg, 27.2 mg, 22.6 mg, and 13.2 mg of substance which was shown by TLC to consist of L-phenylalanine, L-tyrosine, and a trace amount of L-β-3. Elution was then continued with 100 ml of 1.0 N ammonium hydroxide, followed by 75 ml of water. Two fractions, totaling 178 ml, were collected. On evaporation to dryness, these fractions yielded a total of 70.6 mg of substance. TLC revealed that only L-β-3 was present. Since the recovery of L-β-3 was substantially greater than that expected from the reported content of 5.7 mg/gm in the starting material, it is clear that the reported, estimated contents of amino acids in the starting material is an approximation. Nevertheless, the total weight recovered in all fractions from this column, 7.805 gm, was very close to the input of 7.800 gm.

### EXAMPLE 2

### Scale-Up of Fractionation of an Enzymatic Digest of Casein

Employing a column of the same dimensions and prepared in the same manner with AMBERLITE® XAD-4 resin beads as in Example 1, 10.000 gm of the same casein enzymatic hydrolysate was dissolved in 78 ml of water and applied to the column during 21 minutes, followed by elution with water at a flow rate of 3.6 ml/min. Fractions were collected as follows:

| Fraction No. | Color | Volume (ml) |
|---|---|---|
| 1 | yellow | 133.2 |
| 2 | pale yellow | 99.0 |
| 3 | faint yellow | 101.9 |

The eluant was changed to 2% acetic acid in water, 155 ml, followed by elution with water, and the following fractions were collected, all at a flow rate of 3.6 ml/min.

| Fraction No. | Color | Volume (ml) |
|---|---|---|
| 4 | none | 78.5 |
| 5 | none | 91.4 |
| 6 | none | 81.6 |
| 7 | none | 59.4 |

The eluant was changed to 100 ml of 1 N ammonium hydroxide, followed by elution with water at a flow rate of 4.0 to 4.2 ml/min, and the following fractions were collected:

| Fraction No. | Color | Volume (ml) |
|---|---|---|
| 8 | pale straw | 123.3 |
| 9 | none | 45.0 |

Fraction No. 8 was reduced to a thick, pale-yellow syrup by vacuum evaporation at 98° C. To this was added 25 ml of water and the evaporation was repeated to remove the last of the ammonia. On standing for 48 hours at room temperature, the oily residue yielded large, fern leaf-shaped crystals of L-β-3, confirmed by TLC in parallel with pure, authentic substance. Fraction Nos. 8 and 9 together yielded a total of 46.9 mg of L-β-3. Fraction Nos. 4, 5, and 6 together yielded a total of 192 mg of substance which was mostly L-phenylalanine with a lesser amount of L-tyrosine and a trace amount of L-β-3, as shown by TLC analysis.

### EXAMPLE 3

### Adsorptive Separation of L-phenylalanine, L-Tyrosine, and L-β-3 from Non-Aromatic Amino Acids in a Batch Process

240 grams of AMBERLITE® XAD-4 resin was successively pre-washed with 2.0 liters of water, 1.1 liter of 91% isopropyl alcohol, and 5.5 liters of water and then placed in a 1.5 liter beaker equipped with a mechanical stirrer. 78.00 gm of the amino acid mixture was dissolved in 780 ml of water and added to the beaker containing the resin. The mixture was stirred for 1.0 hour at room temperature (22° C.), at a rate sufficient to maintain the resin in a uniform suspension.

The resin was filtered off in a Buchner funnel equipped with a coarse grade of filter paper (Whatman #1). The yellow filtrate was identified as Filtrate No. 1 and stored under refrigeration for later use.

The resin was returned to the beaker, stirred with 250 ml of water for 15 minutes (a slightly longer time does not appear to affect the process), and filtered. The pale yellow filtrate was identified as Filtrate No. 2 and refrigerated for later use.

The water wash was repeated and Filtrate No. 3 was refrigerated.

The resin was returned to the beaker and stirred with 250 ml of 2% aqueous acetic acid for 15 minutes at room temperature. The resin was filtered off and the colorless filtrate was identified as Filtrate No. 4 and preserved under refrigeration for later use.

The resin was returned to the beaker and washed twice, 10 minutes per wash, with 240 ml portions of water, and filtered after each wash. These water washes were identified as Filtrate Nos. 5 and 6, and held under refrigeration for later use.

The resin was returned to the beaker and stirred for 15 minutes with 250 ml of 1 N ammonium hydroxide. The resin was separated by filtration and the pale straw-colored filtrate was identified as Filtrate No. 7.

The resin was returned to the beaker and washed twice, 10 minutes per wash, with 240 ml portions of water, filtering after each wash. The filtrates were identified as Filtrate Nos. 8 and 9, and held under refrigeration for later use.

As Filtrate No. 7 was expected to contain the major portion of the recoverable L-β-3, it was immediately vacuum-evaporated at 98° C., the residue twice re-dissolved in 25 ml portions of water and re-evaporated under vacuum to remove all of the ammonia. The fully dried off-white residue weighed 355 mg (445 mg expected) and was shown by TLC in parallel with a standard to be substantially pure L-β-3 with no more than a trace of L-phenylalanine and no L-tyrosine. When this sample was dissolved/suspended in 5.0 ml of water and refrigerated, it was converted to a crystalline mass over a period of several days. Filtration and drying provided 271.3 mg of substantially pure, crystalline product, L-β-3.

An aliquot of Filtrate No. 1 was vacuum-evaporated to dryness at 98° C. The weight recovered corresponded to a weight of 59.0 gm in Filtrate No. 1. TLC revealed the presence of mixed aliphatic amino acids, no L-phenylalanine, no L-β-3, and a faint trace of L-tyrosine. Therefore the entire Filtrate No. 1 was spray-dried under vacuum and mild heat to produce a pale tan powder weighing (in total) 58.5 gm and possessing a pleasant, slightly meaty flavor with a sweet background note.

Investigation of Filtrate No. 4 revealed that it contained 2.55 gm of solids (dry) which, based upon TLC analysis, consisted of a mixture of L-phenylalanine and a relatively minor amount of L-tyrosine. No other amino acids were apparent in this product.

Finally, Filtrate No. 8 was investigated and found to contain 32 mg of L-β-3 (TLC).

### EXAMPLE 4

### 13% L-β-3 Admixed with Aliphatic Amino Acids by Dry Compounding

A mixture of 13.0 gm of L-β-3 and 87.0 gm of spray-dried aliphatic amino acids (consisting of glycine and the L-forms of alanine, arginine, aspartic acid, cysteine, glutamic acid, histidine, isoleucine, leucine, lysine, methionine, proline, serine, threonine, and valine, obtained, for example, from Filtrate No. 1 of Example 3) was placed in a small ball-mill equipped with ceramic balls, and milled for 4.0 hours at ambient temperature. The resulting fine, light-tan powder was found to have a total nitrogen content of 13.1% and a pleasant, slightly meat-like odor and flavor.

### EXAMPLE 5

### 25% L-β-3 Admixed with Aliphatic Amino Acids by Aqueous Compounding

As was noted in Example 3, Filtrate No. 7 was found to contain 355 mg of L-β-3 in 250 ml of solution and Filtrate No. 1 contained 58.5 gm of mixed aliphatic amino acids in a volume of 780 ml. Therefore, in a fractionation experiment like that of Example 3, the entire Filtrate No. 7, 240 ml, was combined with 14.2 ml of Filtrate No. 1 (which was calculated to contain 1065 mg of mixed aliphatic amino acids) and the entire solution evaporated to dryness under vacuum at 98° C. It was re-evaporated to dryness each time after 2 successive additions of 40 ml of water, until the condensate from the evaporation was neutral, showing that all of the ammonia had been displaced. The remaining dry solid, 1.32 gm, was found on three successive analyses of samples taken randomly from the mixture to have a total nitrogen content of 13.2%, 13.3%, and 13.3%. The total nitrogen content is noticeably greater than that of the starting material, probably because of the formation of ammonium salts of aspartic and glutamic acids in the mixed aliphatic amino acids during exposure to the excess ammonium hydroxide present in Filtrate No. 7.

### EXAMPLE 6

135 ml of washed AMBERLITE® XAD-4 non-ionic resin was placed inside a fine mesh nylon bag. The bag was inserted into a 200 ml plastic tube. 45 grams of hydrolyzed casein powder was dissolved in 175 ml of water. The casein solution was added to the tube. The tube was attached to a horizontal stir motor and rotated slowly for two hours. The protein solution was drained and replaced by 125 ml of water. The water charge was repeated three more times. 125 ml of 2% acetic acid was added three times, followed by three charges of 125 ml of water. 125 ml of 1 N ammonium hydroxide was added twice. These ammonium hydroxide washes were combined as fraction 1. Final washes of 125 ml of 50% ethanol/water and water were combined as fraction 2. After removal of liquid, fraction 1 gave 800 mg of solids, and fraction 2 gave 400 mg. Fraction 1 was tan in color, and fraction 2 was a darker brown.

### EXAMPLE 7

60 ml of AMBERLITE® XAD-4 non-ionic resin was placed in a fine mesh nylon bag. 32 grams of casein powder was dissolved in 250 ml of water. The resin bag was added to the protein solution in a 1000 ml beaker. The solution was stirred for one hour. The protein solution was poured off and replaced with the first water wash of 1000 ml. This was repeated with three more water washes of 1000 ml. Two 500 ml washes of 2% acetic acids were stirred for one hour each, followed by three 1000 ml water washes for one hour each. 320 ml of 1 N ammonium hydroxide solution was stirred for one hour, followed by three 500 ml water washes. The final wash was 500 ml of 50% ethanol/water. 400 mg of light tan solid was isolated from the ammonium hydroxide and ethanol washes.

### EXAMPLE 8

200 mg of dried hydrolysate is combined with deionized water in a tank at 110° F. and agitated to create an approximately 20% solution. The mixture is then heated at 180° F. for a period of minutes to pasteurize the source and/or further dissolve the hydrolysate. The solution is then cooled to room temperature (or below, such as about 65° F.). A suitable solution temperature ranges based on operation dynamics and may be selected in accordance with the teachings herein. In an embodiment of the present invention, a suitable solution temperature may be, for example, 50-90° F., such as 65-75° F.

In this example, the room temperature solution is then added to the top of a two column series both containing a hydrophobic, nonionic resin at 3-3.5 gallons/minute, with the temperature controlled at 80° F. Each column is about 24 inches in diameter and about 8 feet tall. After about 30-45 minutes, the solution has traveled through both columns.

When solids are detected in the output from the second column using a refractometer, along with a rapid increase in conductivity, collection of fraction 1 is commenced. About this time, the hydrolysate tank has been emptied, and thus fresh water (or deionized water) is added to continue collection of fraction 1. At this point, phenylalanine, L-β-3 and some tyrosine should be adsorbed to the resin. The remaining amino acids, and other contents of the solution, should be collected in fraction 1. This process is continued for about 2 hours of collection time. At this point, the solids content is down to about 4% and conductivity is below 0.1 mho.

At this point, the collection is switched to a second tank to begin collecting fraction 2. The flow rate of the water is increased to 9.6-10 gallons/minute to strip phenylalanine from the resin. This will result in some L-β-3, some tyrosine, and some residual aliphatic amino acids being flushed as well. After two hours, solids and conductivity measurements are close to zero.

At this point, the collection is switched to a third tank to begin collection of fraction 3. A 10% isopropyl alcohol solution is added to the columns at a flow rate of 9.6-10 gallons/minute primarily to flush L-β-3. After about 1 hour, the isopropyl alcohol is substituted with water (or deionized water) and continued for one hour to ensure complete, or near complete, dissociation of the remaining L-β-3.

Starting with 200 kg and following the method of the present example may result in approximately 130 kg of final product from fraction 1, approximately 8 kg of final product from fraction 2, and approximately 1 kg of final product from fraction 3.

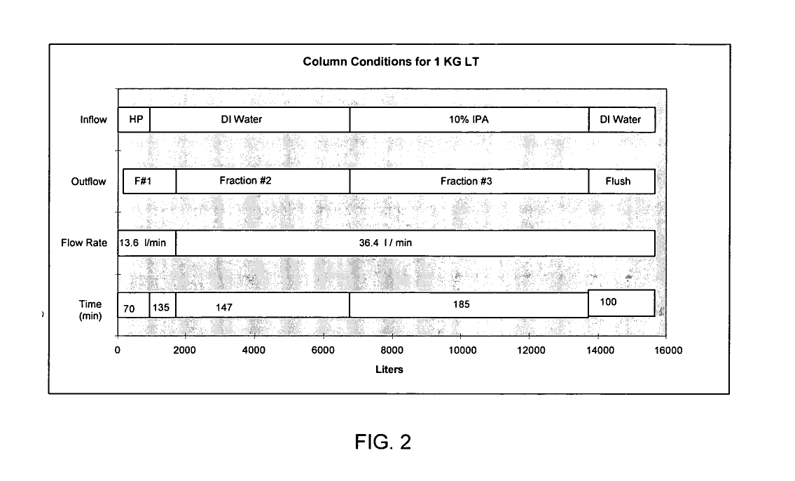

FIG. 2 shows exemplary process conditions for an embodiment of the present invention similar to that described in the present example. In FIG. 2, HP refers to hydrolyzed protein, DI Water refers to deionized water, IPA refers to isopropyl alcohol, and F#1 refers to fraction 1. The relationship among solvent, flow rate, time, and elution volume are depicted.

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

What is claimed is:

1. A process, comprising:
providing an aqueous solution of a mixture of amino acids; and
contacting a hydrophobic substance with the aqueous solution to form a first fraction comprising non-aromatic amino acids, wherein the hydrophobic substance is attractive to aromatic amino acids and has substantially no attraction to non-aromatic amino acids.

2. The process of claim 1, wherein said contacting a hydrophobic substance comprises contacting a polymeric resin.

3. The process of claim 1, wherein said contacting a hydrophobic substance comprises contacting a resin selected from the group consisting of a porous resin, a non-ionic resin, a non-ionic porous resin, a non-ionic cross-linked polystyrene, a particulate-form resin, and a bead-form resin.

4. The process of claim 1, further comprising recovering at least one non-aromatic amino acid from the first fraction.

5. The process of claim 4, wherein said recovering comprises at least one process selected from the group consisting of filtration, reverse osmosis, evaporation, centrifugation, and crystallization.

6. A product recovered by the process of claim 5.

7. The process of claim 1, further comprising flushing the hydrophobic substance to remove residual non-aromatic amino acids from the hydrophobic substance.

8. The process of claim 1, further comprising contacting the hydrophobic substance with a wash agent to remove monocyclic aromatic amino acids from the hydrophobic substance to form a second fraction.

9. The process of claim 8, wherein said contacting the hydrophobic substance with a wash agent comprises contacting the hydrophobic substance with water.

10. The process of claim 8, wherein said contacting the hydrophobic substance with a wash agent comprises contacting the hydrophobic substance with an acid.

11. The process of claim 8, further comprising recovering at least one monocyclic aromatic amino acid from the second fraction.

12. The process of claim 11, wherein said recovering comprises at least one process selected from the group consisting of filtration, reverse osmosis, evaporation, centrifugation, and crystallization.

13. A product recovered by the process of claim 12.

14. The process of claim 8, further comprising combining at least a portion of the first fraction with at least a portion of the second fraction.

15. The process of claim 8, further comprising contacting the hydrophobic substance with a release agent to remove L-β-3 from the hydrophobic substance to form a third fraction.

16. The process of claim 15, further comprising recovering L-β-3 from the third fraction.

17. The process of claim 16, wherein said recovering comprises at least one process selected from the group consisting of filtration, reverse osmosis, evaporation, centrifugation, and crystallization.

18. A product recovered by the process of claim 17.

19. The process of claim 15, further comprising combining at least a portion of the first fraction with a portion of the third fraction.

20. The process of claim 15, wherein said contacting the hydrophobic substance with a release agent comprises contacting the hydrophobic substance with at least one base.

21. The process of claim 15, further comprising flushing the hydrophobic substance to remove residual L-β-3 from the hydrophobic substance.

22. The process of claim 21, wherein said flushing the hydrophobic substance comprises flushing the hydrophobic substance with water.

23. The process of claim 21, wherein said flushing the hydrophobic substance comprises flushing the hydrophobic substance at a flow rate of approximately 8 to 11 gallons per minute.
